(19) United States
(12) Patent Application Publication
(10) Pub. No.: US 2026/0198909 A1
(43) Pub. Date: Jul. 16, 2026

(54) BIOPSY DEVICES AND METHODS OF OPERATING THE SAME

(71) Applicant: BARD PERIPHERAL VASCULAR, INC., Franklin Lakes, NJ (US)

(72) Inventor: Bryon PELZEK, Gold Canyon, AZ (US)

(73) Assignee: BARD PERIPHERAL VASCULAR, INC., Franklin Lakes, NJ (US)

(21) Appl. No.: 19/136,244

(22) PCT Filed: Dec. 9, 2022
(86) PCT No.: PCT/US2022/081223

§ 371 Date: Jun. 5, 2025

(51) Int. Cl. A61B 10/02

(52) U.S. Cl. CPC A61B 10/0275

(57) ABSTRACT

A biopsy device includes a manifold having a first passageway, a second passageway, and a third passageway. A fluid cartridge is operably coupled to the manifold and provides fluid to the manifold. A cutting mechanism is operably coupled to the manifold, and includes a cutting cannula, a sample notch cannula, and a sample storage area. A handheld member is slidably coupled to the manifold, and manual translation of the handheld member directs fluid between the first passageway, the second passageway, and the third passageway.

### TECHNICAL FIELD

The present disclosure relates to biopsy devices, and, more particularly, to handheld single insertion biopsy devices and methods of operating the same.

### BACKGROUND

A typical biopsy device includes a probe assembly having a cannula with a sample notch and a tissue sampling chamber and associated tissue cutting mechanism. Some biopsy devices, commonly referred to as single insertion, multiple samples, or SIMS devices, utilize sample acquisition and delivery mechanisms that allow multiple samples to be acquired from a given lesion without removing and reinserting the needle after each sample. However, these devices are often used in connection with large console systems that are difficult to transport and are reliant on electricity to operate.

### SUMMARY

An object of the present disclosure is to provide a biopsy device that can perform biopsy procedures off-grid and under circumstances where electric power is limited or unavailable.

In one embodiment, a biopsy device is disclosed. The biopsy device may include a manifold having a first passageway, a second passageway, and a third passageway, and a fluid cartridge may be operably coupled to the manifold for providing fluid to the manifold. The biopsy device may further include a cutting mechanism operably coupled to the manifold, a sample notch, and a sample storage area. A handheld member may be slidably coupled to the manifold, and may direct fluid between the first passageway, the second passageway, and the third passageway.

In another embodiment, a biopsy device is disclosed. The biopsy device may include a manifold having a first passageway, a second passageway, and a third passageway, and a fluid cartridge may be operably coupled to the manifold for providing fluid to the manifold. The biopsy device may further include a cutting mechanism operably coupled to the manifold, a sample notch, and a sample storage area. A handheld member may be slidably coupled to the manifold, and may direct fluid between the first passageway, the second passageway, and the third passageway. The first passageway of the manifold may comprise a first valve for creating a vacuum to introduce the tissue sample into the sample notch. The second passageway of the manifold may include a second valve for maintaining the vacuum in the sample notch and pushing the cutting cannula forward across the tissue sample. The third passageway may include a pneumatic cylinder which retracts the cutting mechanism and transport the tissue sample to the storage area.

In yet another embodiment, a method of operating a biopsy device is disclosed. The method may involve providing fluid to a manifold of the biopsy device using a fluid cartridge, and manually sliding a handheld member of the biopsy device to a first position in the manifold. The method may then involve creating a vacuum for capturing a tissue sample in the biopsy device. With the tissue sample captured, the method may involve manually sliding a handheld member of the biopsy device to a second position in the manifold, and pushing a cutting mechanism of the biopsy device across the tissue sample. The method may then involve manually sliding a handheld member of the biopsy device to a third position, and retracting the cutting mechanism of the biopsy device such that the tissue sample is returned to a sample acquisition area of the biopsy device. The cylindrical slider may then be returned to the first position of the biopsy device.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a longitudinal side view of an illustrative biopsy device in a first position, according to one or more embodiments shown and described herein;

FIG. 2 depicts a longitudinal side view of the biopsy device of FIG. 1 in a second position, according to one or more embodiments shown and described herein;

FIG. 3 depicts a longitudinal side view of the biopsy device of FIG. 1 in a third position, according to one or more embodiments shown and described herein; and

FIG. 4 depicts a flow chart of an illustrative method of performing a biopsy procedure using the biopsy device of FIG. 1, according to one or more embodiments shown and described herein;

Reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

Embodiments disclosed herein relate to biopsy devices and methods of performing biopsy procedures. For example, in embodiments, a biopsy device includes a manifold, a vacuum assembly, and a cutting mechanism. A handheld member, such as a cylindrical slider, having a first passageway, a second passageway, and a third passageway may be slidably coupled to the manifold, such that movement of the cylindrical slider through the manifold directs a fluid through the various passageways and into the vacuum assembly and cutting mechanism. The cutting mechanism may include a cutting cannula, a sample notch, and a sample storage area. The vacuum assembly may provide a vacuum to the cutting mechanism for obtaining a tissue sample. When the cylindrical slider is in a first position, fluid may pass through the first passageway and into the vacuum assembly such that the vacuum assembly provides a vacuum which draws a tissue sample into the sample notch of the cutting mechanism. When the cylindrical slider is in a second position, fluid may pass through the second passageway and into the vacuum assembly and cutting mechanism simultaneously. In the second position, the fluid may maintain the vacuum in the vacuum assembly while also serving to advance the cutting cannula of the cutting mechanism towards the tissue sample. When the cylindrical slider is in a third position (e.g., once the cutting cannula has advanced over and severed the tissue sample), fluid may pass through the third passageway and to the cutting mechanism. In the third position, the fluid may act to retract the cutting cannula of the cutting mechanism.

Embodiments of the present disclosure may be specifically advantageous for performing biopsy procedures in scenarios where traditional console-based biopsy devices are inconvenient, unavailable, or inoperable. The use of the manifold, such as a pneumatic air manifold, may allow a plurality biopsy samples to be obtained from a target area by utilizing a pressurized fluid, such as air or any other suitable fluid, to operate the biopsy device. Furthermore, the cylindrical slider coupled to the manifold may allow the biopsy procedure to be performed by manually alternating the flow of the pressurized fluid between the first passageway, second passageway, and third passageway of the manifold.

Embodiments of biopsy devices, and methods of performing biopsy procedures will now be described in more detail herein with reference to the drawings and where like numbers refer to like structures.

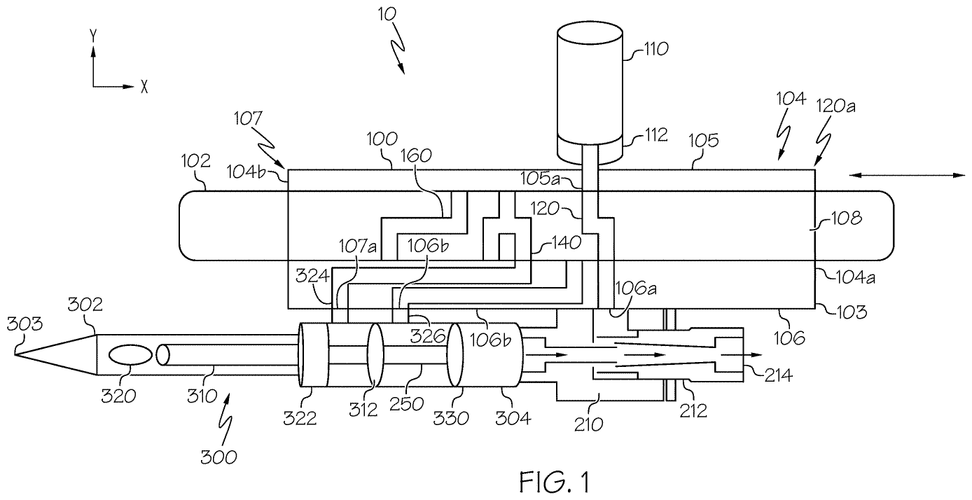

Referring now to FIGS. 1-3, a biopsy device 10 is depicted. The biopsy device may include a manifold 100, a vacuum assembly 200, and a cutting mechanism 300. The manifold 100 may be a pneumatic manifold that directs air flow to the vacuum assembly 200 and the cutting mechanism 300. In some embodiments, a fluid cartridge 110, such as a carbon dioxide cartridge, may be coupled to the manifold 100 such that a fluid, such as air or any other suitable fluid, may be used to operate the biopsy device 10. In these embodiments, the fluid cartridge 110 may be coupled to the manifold 100 via a fluid supply valve 112. The fluid supply valve 112 may include any suitable valve which is capable of selectively allowing fluid to pass from the fluid cartridge 110 and into the manifold 100. The fluid cartridge 110 and manifold 100 may be connected to the fluid supply valve 112 using any suitable connection (e.g., via threaded connection, adhesive, welding, brazing, etc.).

The fluid supply valve 112 may alternate between an open position and a closed position. In the open position, fluid may pass from the fluid cartridge 110 and into the manifold 100 in order to perform various biopsy processes, as will be described in more detail herein. In the closed position, the fluid supply valve 112 may prevent fluid from the fluid cartridge 110 from passing into the manifold 100, such that any pressure or vacuum within the biopsy device 10 may be alleviated. In some embodiments, the fluid supply valve 112 may be manually rotated between the open position and the closed position, such that a user may easily control the supply of fluid to the biopsy device 10. In these embodiments, the fluid supply valve 112 may further include a switch, such as a trigger switch, which may be engaged to prevent the flow of fluid through the fluid supply valve 112 while a user manually moves the fluid supply valve 112 between the open position and the closed position. The trigger switch may be coupled to the fluid supply valve 112, such that engagement of the trigger switch blocks the flow of fluid from the fluid cartridge 110

Although FIGS. 1-3 depict a biopsy device 10 having a single fluid cartridge 110, it should be understood that additional fluid cartridges may be used in connection with the biopsy device 10. For example, in some embodiments, the biopsy device 10 may include a second fluid cartridge 110, which may be used to supply an additional source of pressure to the biopsy device 10. In these embodiments, the work needed to operate the biopsy device 10 may be divided amongst multiple fluid cartridges 110, such that each of the multiple fluid cartridges 110 may be capable of providing pressure for a different component of the biopsy device 10. Although the fluid cartridge 110 illustrated in FIGS. 1-3 may be a carbon dioxide cartridge, it should be understood that the biopsy device may utilize any type of pressurized fluid cartridge sufficient for operating the biopsy device 10. For example, a pressurized helium cartridge, a pressurized argon cartridge, a pressurized nitrogen cartridge, or the like, may be similarly used to operate the biopsy device 10.

Referring still to FIGS. 1-3, the manifold 100 may include a body 104 having a proximal end 104a and a distal end 104b. In these embodiments, the body 104 may define a central opening 108, which may extend in a longitudinal direction (e.g., in the +/−x direction of the coordinate axes of FIGS. 1-3) between the proximal end 104a and the distal end 104b of the body 104. The body 104 of the manifold 100 may further include a first side wall 103 on the proximal end 104a, a second side wall 107 opposite the first side wall 103 on the distal end 104b, a top surface 105 and a bottom surface 106 opposite the top surface 105. The top surface 105 may include an opening 105a, which may receive the fluid supply valve 112 of the fluid cartridge 110 such that fluid from the fluid cartridge 110 passes into the manifold 100. In these embodiments, the opening 105a may be in fluid communication with the central opening 108 such that fluid from the fluid cartridge 110 enters the central opening 108 of the manifold 100.

Referring again to FIGS. 1-3, the biopsy device 10 may further include a handheld member 102. The handheld member 102 may be received by the central opening 108 of the manifold 100, such that the handheld member 102 is slidably coupled to the manifold 100. In these embodiments, the handheld member 102 may be inserted into either the distal end 104b or proximal end 104a of the body 104 of the manifold and into the central opening 108.

The handheld member 102 may further include a plurality of passageways, such as a first passageway 120, a second passageway 140, and a third passageway 160. Although the biopsy device 10 illustrated in FIGS. 1-3 includes three passageways, it should be understood that the number of the plurality of passageways may vary without going beyond the scope of the present disclosure. For example, the biopsy device may include two passageways, four passageways, or any other number of passageways required to perform various steps of a desired biopsy procedure.

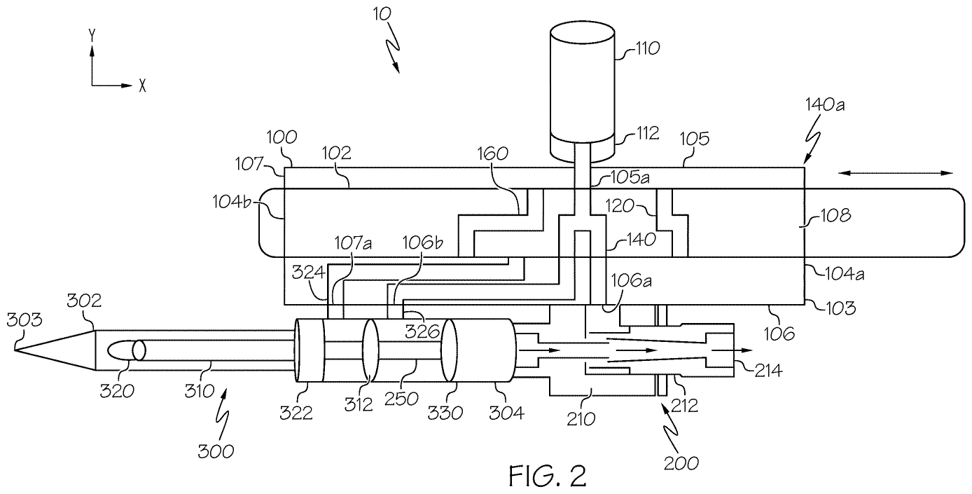

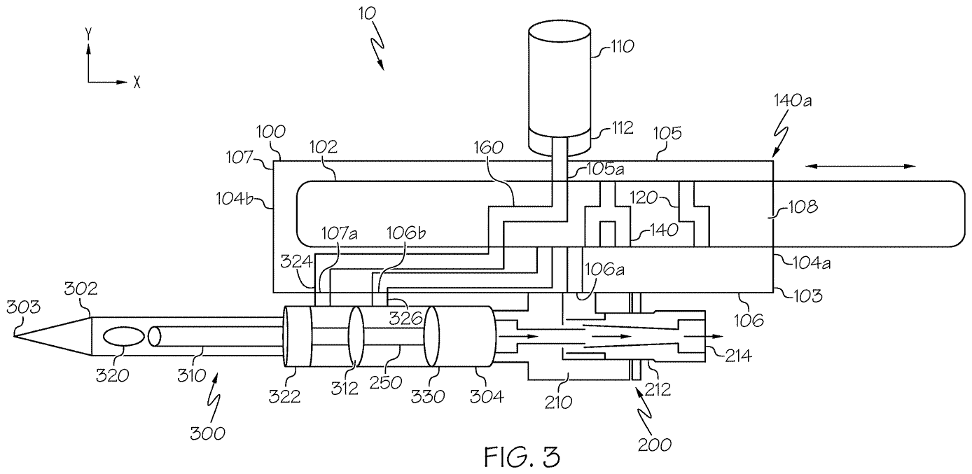

The handheld member 102 may further be movable within the central opening 108 of the manifold 100 between a plurality of positions that may coincide with the plurality of passageways of the handheld member 102. For example, as illustrated in FIGS. 1-3, the handheld member 102 may be manually alternated between a first position 120a (FIG. 1), a second position 140a (FIG. 2), and a third position 160a (FIG. 3). In these embodiments, the passageway 120, 140, 160 of the handheld member 102 corresponding to the respective position 120a, 140a, 160a may be aligned with the valve 112 to direct fluid from the fluid cartridge 110 to the corresponding passageway 120, 140, 160. That is, in the first position 120a, the handheld member 102 is positioned such that the valve 112 is aligned with the first passageway 120 to direct fluid from the fluid cartridge 110 to the first passageway 120 of the handheld member 102. In these embodiments, the first passageway 120 of the handheld member 102 may pass fluid from the fluid cartridge 110 to the vacuum assembly 200 of the biopsy device 10.

In the second position 140a, the handheld member 102 is positioned such that the valve 112 is aligned with the second passageway 140 to direct fluid from the fluid cartridge 110 to the second passageway 140 of the handheld member 102. In these embodiments, the second passageway 140 of the handheld member 102 may pass fluid from the fluid cartridge 110 to the vacuum assembly 200 and the cutting mechanism 300 of the biopsy device 10 simultaneously. In order to allow fluid supply to both the vacuum assembly 200 and the cutting mechanism 300 simultaneously, the second passageway 140 may include a valve, such as a y-valve, or any other suitable valve capable of supplying fluid to both components of the biopsy device 10 at the same time.

In the third position 160a, the handheld member 102 is positioned such that the valve 112 is aligned with the third passageway 160 to direct fluid from the fluid cartridge 110 to the third passageway 160 of the handheld member. In these embodiments, the third passageway 160 of the handheld member 102 may pass fluid from the fluid cartridge 110 to the cutting mechanism 300 of the biopsy device 10.

Although the biopsy device 10 depicted in FIGS. 1-3 illustrates the handheld member 102 alternating between three positions, it should be understood that the handheld member 102 may be moved between any number of positions, such that the plurality of positions of the handheld member 102 correspond to the number of plurality of passageways present in the manifold 100. Furthermore, the trigger switch, which may be used to disassociate functionality of the fluid cartridge 110, may be engaged while the handheld member 102 is moved between the plurality of positions 120a, 140a, 160a, such that fluid is not being supplied to the biopsy device 10 while the handheld member 102 is alternated.

It should be further noted that the handheld member 102 may take a variety of different shapes, so long as the central opening 108 of the manifold 100 corresponds to the shape of the member 102. For example, the handheld member 102 may be a rectangular member. In these embodiments, the central opening 108 may have a rectangular shape, such that the central opening can receive the member and allow the member to slide in the longitudinal direction through the manifold 100.

In the embodiments described herein, the manifold 100 and/or handheld member 102 may be made of a plastic material, such as polycarbonate. In some embodiments, the manifold 100 and/or handheld member 102 may be made of a transparent or translucent plastic material that allows a user to readily identify the position of the handheld member 102 within the manifold 100. By identifying the position of the handheld member 102 within the manifold 100, a user may ensure that the handheld member 102 is appropriately aligned within the manifold 100, and is prepared to perform a desired biopsy process. Rather than forming the entire manifold 100 and/or handheld member 102 from transparent or translucent plastic, other embodiments may include a transparent or translucent window along some portion of the manifold 100 and/or handheld member 102 that provides a view at one or more discrete points. However, it is to be appreciated that the manifold 100 and/or handheld member 102 may be made entirely of an opaque material, as the present disclosure is not limited to include translucent or transparent materials. In these embodiments, the manifold 100 and/or handheld member 102 may include a plurality of hash lines, or any other similar indicia or visual indicator, which may allow a user to ensure that the handheld member 102 is appropriately positioned within the manifold 100.

Referring still to FIGS. 1-3, the vacuum assembly 200 and cutting mechanism 300 may be operably coupled to the manifold 100 such that fluid from the fluid cartridge 110 may pass through the plurality of passageways 120, 140, 160 and into the vacuum assembly 200 and cutting mechanism 300, respectively, as will be described in more detail herein.

The vacuum assembly 200 may include a first valve 210, which may engage the bottom surface 106 of the manifold such that the vacuum assembly 200 is in fluid communication with the central opening 108 of the manifold. In these embodiments, the bottom surface 106 may further include a first opening 106a which may receive the first valve 210. The first valve 210 may engage the first opening 106a of the bottom surface 106 of the manifold 100 via any suitable connection (e.g., via threaded connection, adhesive, welding, brazing, etc.).

By coupling the first valve 210 to the manifold 100, fluid from the fluid cartridge 110 may enter the vacuum assembly 200 when the handheld member 102 is in the first position 120a or the second position 140a, as described herein. As fluid enters the first valve 210, a restriction within the first valve 210 may act to increase the speed of the flow of the fluid. As the speed of the flow of the fluid increases, the fluid pressure may decrease, which may result in a vacuum being generated within the vacuum assembly 200. In these embodiments, the first valve 210 may be a venturi valve, but it should be understood that the first valve 210 may include any valve suitable to generate a vacuum within the vacuum assembly 200.

The vacuum assembly 200 may further include a rotational valve 212 coupled to the first valve 210, which may be used to control the vacuum generated by the first valve 210. For example, rotating the rotational valve 212 in a first direction may decrease the amount of fluid expelled from the vacuum assembly 200 as exhaust, thereby decreasing the pressure of the fluid and increasing the vacuum generated in the vacuum assembly 200. Similarly, rotating the rotational valve 212 in a second direction may increase the amount of fluid expelled from the vacuum assembly 200 as exhaust, thereby increasing the pressure of the fluid and decreasing the amount of vacuum generated in the vacuum assembly 200.

Although the first valve 210 and the rotational valve 212 are depicted as being in a horizontal position (e.g., positioned parallel to the x-axis depicted in FIGS. 1-3), it should be noted that, in some embodiments, the first valve 210 and the rotational valve 212 may be aligned in a vertical position (e.g., positioned perpendicularly to the x-axis depicted in FIGS. 1-3). Furthermore, in these embodiments, the first valve 210 and the rotational valve 212 may be rotated between the horizontal position and the vertical position, which may allow a user to more easily control the vacuum generated by the first valve 210.

Referring still to FIGS. 1-3, the vacuum assembly 200 may be further coupled to the cutting mechanism 300 such that the vacuum assembly 200 and the cutting mechanism 300 are in fluid communication with one another. In these embodiments, the cutting mechanism 300 may include a distal end 302 and a proximal end 304, with the proximal end 304 being coupled to the first valve 210 of the vacuum assembly 200. The first valve 210 and proximal end 304 of the cutting mechanism 300 may be coupled via any suitable connection (e.g., via threaded connection, adhesive, welding, brazing, etc.) such that vacuum generated within the vacuum assembly 200 may be used to operate components of the cutting mechanism 300. For example, a fluid pathway 250 between the cutting mechanism 300 and the vacuum assembly 200 may allow the vacuum generated by the vacuum assembly 200 to act on the cutting mechanism 300.

The cutting mechanism 300 may also be fluidly coupled to the manifold 100 by way of a first fluid tube 324 and a second fluid tube 326 such that fluid may pass from the fluid cartridge 110 and into the cutting mechanism 300 when the handheld member 102 is in either the second position 140a or the third position 160a. As illustrated in FIGS. 1-3, the manifold may include a second opening 106b in the bottom surface 106 of the manifold, which may receive the second fluid tube 326. The second side wall 107 may further include a first opening 107a, which may receive the first fluid tube 324. The first fluid tube 324 and the second fluid tube 326 may be coupled to the manifold 100 via any suitable connection (e.g., via threaded connection, adhesive, welding, brazing, etc.) such that fluid may pass through the second fluid tube 326 when the handheld member 102 is in the second position 140a, and fluid may pass through the first fluid tube 324 when the handheld member 102 is in the third position 160a.

Referring still to FIGS. 1-3, the distal end 302 of the cutting mechanism may include a piercing tip 303, which may be used to provide access to a target area. For example, the piercing tip 303 may include a sharpened edge, such that the piercing tip 303 is able to penetrate areas of tissue and access tissue samples located at a target area. The cutting mechanism 300 may further include a cutting cannula 310, a sample notch 320, and a sample storage area 330, which may obtain a tissue sample from a target area during a biopsy procedure. The sample notch 320 may extend in a longitudinal (e.g., in the +/−x direction of the coordinate axes of FIGS. 1-3) direction, and may receive a tissue sample when vacuum is applied to the cutting mechanism 300. A dial 322, such as a rotational dial, may be coupled to the cutting mechanism 300, and may be used to rotate the sample notch 320 in a circumferential direction about its axis. In these embodiments, the dial 322 may be fixedly attached to the sample notch 320 and rotatably attached to the sample storage area 330, such that rotation of the dial 322 results in rotation of the sample notch 320. The dial 322 may further be manually operated, such that the rotational position of the sample notch 320 may be easily adjusted by rotating the dial 322. By rotating the sample notch 320, it may be possible to obtain multiple tissue samples from a target area without the need to remove and reinsert the biopsy device 10, as the sample notch may be able to obtain tissue samples from areas around the complete circumference of the target area.

Once the tissue sample has been drawn into the sample notch 320, fluid from the fluid cartridge 110 may be supplied through the first fluid tube 324 to the cutting mechanism 300 such that the cutting cannula 310 advances in a longitudinal (e.g., in the −x direction of the coordinate axes of FIGS. 1-3) direction across the tissue sample drawn into the sample notch 320 by moving the handheld member 102 to the second position 140a. In these embodiments, the cutting cannula 310 may include a base 312, which may be used to move the cutting cannula 310 in the longitudinal (e.g., in the +/−x direction of the coordinate axes of FIGS. 1-3) direction. For example, in order to advance the cutting cannula 310 (e.g., towards the distal end 302 of the cutting mechanism 300 in the −x direction of the coordinate axes of FIGS. 1-3), fluid may be supplied from the fluid cartridge 110, through the second fluid tube 326, and into a space between the base 312 of the cutting cannula 310 and the sample storage area 330. As the fluid fills the space between the base 312 of the cutting cannula 310 and the sample storage area 330, the pressure of the fluid may advance the cutting cannula 310 in the longitudinal (e.g., in the −x direction of the coordinate axes of FIGS. 1-3) direction across the tissue sample drawn into the sample notch 320. As the cutting cannula 310 moves across the tissue sample drawn into the sample notch 320, the cutting cannula 310 may sever the tissue sample from the target area. In these embodiments, the cutting cannula 310 may have a cutting edge for severing the tissue sample from the target area.

With the tissue sample severed from the target area, the vacuum acting within the fluid pathway 250 transports the tissue sample through the cutting cannula 310 and into the sample storage area 330. The sample storage area 330 may store excised tissue transported through the cutting mechanism 300 until the desired number of tissue samples have been excised. Once the desired number of tissue samples are obtained, the fluid cartridge 110 may be deactivated by turning the fluid supply valve 112 to the closed position, and the tissue samples may be collected from the sample storage area 330.

The cutting cannula 310 may be retracted once the tissue sample has successfully traversed the fluid pathway 250 to the sample storage area 330. In order to retract the cutting cannula 310 in a retracted direction (e.g., towards the proximal end 304 of the cutting mechanism 300 in the +x direction of the coordinate axes of FIGS. 1-3), the handheld member 102 may be moved to the third position 160a, such that fluid may be supplied from the fluid cartridge 110 to an area in between the base 312 of the cutting cannula 310 and the dial 322. As the fluid fills the area in between the base 312 of the cutting cannula 310 and the dial 322, the pressure of the fluid may retract the cutting cannula 310 in the retracted direction, such that the cutting cannula 310 exposes the sample notch 320 and another tissue sample may be obtained.

In some embodiments, the biopsy device 10 may further include a firing mechanism, which may be used to rapidly propel the piercing tip 303 of the cutting mechanism 300 into an area of dense tissue. In these embodiments, the firing mechanism may be a pneumatic firing mechanism or a spring-loaded firing mechanism. In embodiments in which the firing mechanism is a pneumatic firing mechanism, the manifold 100 may include a fourth passageway, which may be used to provide fluid from the fluid cartridge 110 to the firing mechanism. The fourth passageway may include a fourth valve, which may be operably coupled to the cutting mechanism 300 to store compressed fluid provided from the fluid cartridge 110. Once a desired amount of fluid has built within the fourth valve, the fluid may be released, such as via a button, switch, or other trigger mechanism. As the fluid is released from the fourth valve, the pressurized fluid may propel the cutting mechanism 300 forward.

Similar embodiments may utilize the spring-loaded firing mechanism in place of the pneumatic firing mechanism. In these embodiments, a spring assembly may be operably coupled to the cutting mechanism 300, such that the cutting mechanism 300 may move in a longitudinal direction in unison with a spring of the spring assembly. The spring may then be compressed to a cocked position to prime the cutting mechanism 300. Once the biopsy device 10 is positioned at a target area, the spring may be released, such as a by a button, switch, or other trigger mechanism, which may cause the cutting mechanism 300 and piercing tip 303 to be fired into the target area.

Referring to FIG. 1, the biopsy device 10 is illustrated with the handheld member 102 in the first position 120a within the manifold 100. In the first position 120a, the handheld member 102 may be aligned with the first passageway 120 of the manifold 100, such that fluid from the fluid cartridge 110 passes through the first passageway 120 and into the first valve 210. As the fluid passes into the first valve 210 a vacuum is generated, as described herein. The vacuum may pull fluid from the sample notch 320 through the fluid pathway 250 and into the vacuum assembly 200, such that the vacuum generated by the first valve 210 may act in the sample notch 320. The negative pressure generated by the vacuum assembly may cause a tissue sample to be drawn into the sample notch 320. In these embodiments, the first valve 210 may further include a filter 214, which may prevent any biological particles transported through the fluid pathway 250 and into the vacuum assembly 200 from being expelled into an external environment.

In some embodiments, the first valve 210 may further include a recirculation mechanism 216. The recirculation mechanism 216 may recirculate exhaust fluid, such as air, which is expelled by the vacuum assembly 200 into the manifold 100. In these embodiments, the exhaust fluid expelled by the vacuum assembly 200 may be collected by the recirculation mechanism 216 and redirected from the vacuum assembly 200 to the first passageway 120 of the manifold 100. By redirecting the exhaust fluid into the first passageway 120 of the manifold 100, the exhaust fluid may be reused to enhance vacuum levels within the vacuum assembly 200.

Referring still to FIG. 1, the handheld member 102 may be maintained in the first position 120a in the manifold 100 until the vacuum generated by the vacuum assembly 200 draws a tissue sample into the sample notch 320 of the cutting mechanism 300. In the event the vacuum generated is insufficient to draw tissue into the sample notch 320, the rotational valve 212 of the first valve 210 may be rotated in the first direction in order to further decrease the fluid pressure applied to the sample notch 320, as described herein.

Referring now to FIG. 2, the biopsy device is illustrated with the handheld member 102 in the second position 140a within the manifold 100. Once the tissue sample has been drawn into the sample notch 320, the handheld member 102 may be manually moved in a longitudinal (e.g., in the −x direction of the coordinate axes of FIG. 2) direction from the first position 120a to the second position 140a, such as by using a thumb or index finger. If desired, a user may engage the trigger switch on the fluid supply valve 112 to disassociate the functionality of the biopsy device 10 as the handheld member 102 is moved from the first position 120a to the second position 140a. The handheld member 102 may be moved in the longitudinal direction until the handheld member 102 is properly aligned with the second passageway 140, such that fluid may pass from the fluid cartridge 110 and into the second passageway 140.

As illustrated in FIG. 2, the second passageway 140 be used to simultaneously supply fluid from the fluid cartridge 110 to the cutting mechanism 300 and the vacuum assembly 200, as described herein. In these embodiments, the fluid flowing from the fluid cartridge may be divided by the y-valve of the second passageway 140, such that a first portion of the fluid may allow the first valve 210 of the vacuum assembly 200 to continue to provide vacuum to the sample notch 320, while a second portion of the fluid may be used to activate the cutting mechanism 300.

As further illustrated in FIG. 2, the second portion of the fluid may traverse the first fluid tube 324 and enter a space between the base 312 of the cutting cannula 310 and the sample storage area 330. As the second portion of the fluid fills the space between the base 312 of the cutting cannula 310 and the sample storage area 330, the pressure of the fluid may advance the cutting cannula 310 in the longitudinal (e.g., in the −x direction of the coordinate axes of FIG. 2) direction across the tissue sample drawn into the sample notch 320. The fluid supplied to the area between the base 312 of the cutting cannula 310 and the sample storage area 330 may continue to propel the cutting cannula 310 in the longitudinal direction (e.g., in the −x direction of the coordinate axes of FIG. 2) until the cutting cannula 310 has moved completely past the sample notch 320. As the cutting cannula 310 moves over and past the sample notch 320, the cutting cannula 310 may sever the tissue drawn into the sample notch 320 from the target area.

While the second portion of the fluid pushes the cutting cannula 310 across the sample notch 320, the first portion of the fluid may continue to flow through the first valve 210 of the vacuum assembly 200, such that the vacuum generated by the vacuum assembly 200 in the first position 120a may be maintained. Because the vacuum is maintained, when the tissue sample is severed from the target area by the cutting cannula 310, the vacuum may act to pull the severed tissue sample through the fluid pathway 250 and into the sample storage area 330.

In these embodiments, the cutting mechanism 300 may be made of a translucent or transparent material, such that a user is able to identify the location of the severed tissue sample within the cutting mechanism 300 without the need to remove the biopsy device 10. As a result, a user may be able to identify if a tissue sample becomes lodged in the fluid pathway 250 and/or the cutting cannula 310. In the event a tissue sample becomes lodged prior to reaching the sample storage area 330, a user may attempt to increase the vacuum generated by the vacuum assembly 200 to aid in transporting the tissue sample to the sample storage area 330. Furthermore, in instances in which the vacuum is insufficient to dislodge the tissue sample, a dry tap may be performed until the tissue sample is transported to the sample storage area 330.

Referring now to FIG. 3, the biopsy device is illustrated with the handheld member 102 in the third position 160a within the manifold 100. Once the tissue sample has been severed by the cutting cannula 310 and transported to the sample storage area 330, the handheld member 102 may be manually moved in the longitudinal (e.g., in the −x direction of the coordinate axes of FIG. 3) direction from the second position 140a to the third position 160a, such as by using a thumb or index finger. If desired, a user may engage the trigger switch on the fluid supply valve 112 to disassociate the functionality of the biopsy device 10 as the handheld member 102 is moved from the second position 140a to the third position 160a. The handheld member 102 may be moved in the longitudinal direction until the handheld member 102 is properly aligned with the third passageway 160, such that fluid may pass from the fluid cartridge 110 and into the third passageway 160.

The third passageway 160 may comprise a third valve, which may be used to direct fluid from the fluid cartridge 110 through the second fluid tube 326 and into the cutting mechanism 300. When the handheld member 102 is in the third position 160a, fluid may only flow to the cutting mechanism 300, such that the vacuum generated by the vacuum assembly 200 is alleviated. The third passageway 160 may be positioned such that fluid flows from the fluid cartridge and into a space between the base 312 of the cutting cannula 310 and the dial 322. As the fluid fills the space between the base 312 of the cutting cannula 310 and the dial 322, the pressure of the fluid may drive the cutting cannula 310 in the backwards (e.g., in the +x direction of the coordinate axes of FIG. 3) direction, such that the cutting cannula 310 retracts and exposes the sample notch 320. With the cutting cannula 310 fully retracted, the handheld member 102 may be manually returned to the first position 120a within the manifold 100, such that additional tissue samples may be maintained. As the handheld member 102 moves between the third position 160a and the first position 120a, the user may engage the trigger switch on the fluid supply valve 112 to disassociate the functionality of the biopsy device 10.

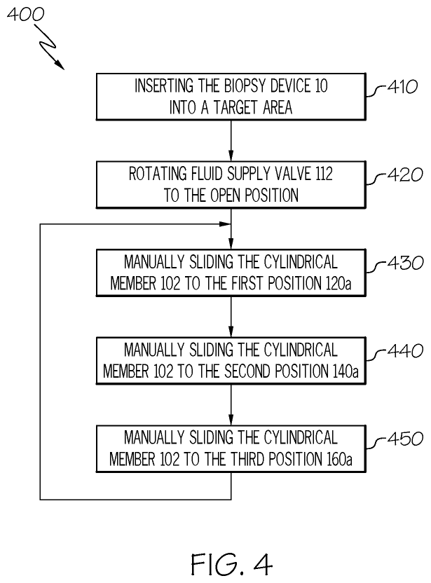

Referring now to FIG. 4, an illustrative method 400 of performing a biopsy procedure is provided. Initially, as shown at block 410, the method may involve inserting the piercing tip 303 of the cutting mechanism 300 into a target area. The piercing tip 303 may be used to create a puncture in the target area such that the biopsy device 10 may be positioned to obtain a tissue sample.

With the biopsy device 10 positioned in the target area, the method may move to block 420, which may involve rotating the fluid supply valve 112 to the open position, such that fluid may begin flowing from the fluid cartridge 110. The method may then move to block 420, which may involve manually sliding the handheld member 102 to the first position 120a and creating a vacuum for drawing a tissue sample into the sample notch 320 of the biopsy device 10.

Once the vacuum has drawn the tissue sample into the sample notch 320, the handheld member 102 may be moved to the second position 140a, as shown at block 430. In the second position 140a, the fluid from the fluid cartridge 110 may be directed through the second passageway 140 and into the cutting mechanism 300, such that the fluid pushes the cutting cannula 310 forward across the tissue sample drawn into the sample notch 320. The cutting cannula 310 may sever the tissue sample from the target area as the cutting cannula 310 moves across the sample notch 320.

The method may next move to block 440, which may involve moving the handheld member 102 to a third position 160a. In the third position 160a, the fluid from the fluid cartridge 110 may be directed through the third passageway 160 and into the cutting mechanism 300, such that the fluid pushes the cutting cannula 310 backwards to expose the sample notch 320 and retract the cutting cannula 310. Once the cutting cannula 310 is fully retracted, the method may move to block 450, which may involve returning the handheld member 102 to the first position 120a. The method 400 may then return to block 410, where the biopsy procedure may be repeated to acquire additional tissue samples from a target area.

Embodiments may be further described with references to the following numbered clauses:
1. A biopsy device comprising: a manifold having a first passageway, a second passageway, and a third passageway; a fluid cartridge operably coupled to the manifold for providing fluid to the manifold; a cutting mechanism operably coupled to the manifold, the cutting mechanism comprising a cutting cannula, a sample notch, and a sample storage area; and a handheld member slidably coupled to the manifold, wherein manual translation of the handheld member directs fluid between the first passageway, the second passageway, and the third passageway.
2. The biopsy device of item 1, wherein the first passageway of the manifold comprises a first valve for creating a vacuum to introduce the tissue sample into the sample notch.
3. The biopsy device of item 2, wherein the first valve comprises a rotational valve which may be manually rotated to control the vacuum created by the first valve.
4. The biopsy device of item 2, wherein the first valve comprises a venturi valve.
5. The biopsy device of any of items 1-3, wherein the, the second passageway of the manifold comprises a second valve for maintaining a vacuum in the sample notch and pushing the cutting cannula forward across the tissue sample.
6. The biopsy device of item 5, wherein the second valve is a y-valve.
7. The biopsy device of any of items 1-6, wherein the third passageway comprises a third valve for retracting the cutting mechanism and transporting the tissue sample to the sample storage area.
8. The biopsy device of any of items 1-7, wherein the handheld member may be manually alternated between the first passageway, second passageway, and third passageway multiple times to obtain multiple tissue samples.
9. The biopsy device of any of items 1-8, wherein the fluid cartridge is a carbon dioxide cartridge.
10. The biopsy device of any of items 1-9, wherein the handheld member, cutting mechanism, and manifold are made of a transparent material.
11. The biopsy device of any of items 1-10, wherein the manifold further comprises a fourth passageway, the fourth passageway pneumatically propels the cutting cannula into an area of dense tissue.
12. The biopsy device of any of items 1-11 wherein the cutting mechanism further comprises a spring mechanism which propels the cutting cannula into an area of dense tissue.
13. The biopsy device of any of items 1-12, further comprising a switch to temporarily disable the biopsy device as the handheld member slides between the third passageway and the first passageway.
14. The biopsy device of any of items 1-13, further comprising a second fluid cartridge operably coupled to the manifold to provide fluid to the manifold.
15. A biopsy device comprising: a manifold having a first passageway, a second passageway, and a third passageway; a fluid cartridge operably coupled to the manifold for providing air to the manifold; a cutting mechanism operably coupled to the manifold, the cutting mechanism comprising a cutting cannula, a sample notch, and a sample storage portion; and a handheld member slidably coupled to the manifold, such that manual translation of the handheld member directs fluid between the first passageway, the second passageway, and the third passageway; wherein the first passageway of the manifold comprises a first valve for creating a vacuum to introduce the tissue sample into the sample notch, the second passageway of the manifold comprises a second valve for maintaining the vacuum in the sample notch and pushing the cutting cannula forward across the tissue sample, and the third passageway comprises a pneumatic cylinder which retracts the cutting mechanism and transports the tissue sample to the sample storage area.
16. A method of operating a biopsy device comprising: providing fluid to a manifold of the biopsy device using a fluid cartridge; manually sliding a handheld member of the biopsy device to a first position in the manifold; creating a vacuum for capturing a tissue sample in the biopsy device; manually sliding a handheld member of the biopsy device to a second position in the manifold; pushing a cutting mechanism of the biopsy device across the tissue sample; manually sliding a handheld member of the biopsy device to a third position; retracting the cutting mechanism of the biopsy device such that the tissue sample is returned to a sample acquisition area of the biopsy device; and returning the handheld member of the biopsy device to the first position, wherein the method may be repeated to obtain additional tissue samples.
17. The method of item 16, further involving creating a vacuum for capturing the tissue sample in the biopsy device using a venturi valve.
18. The method of item 16 or 17, further involving firing the cutting mechanism of the biopsy device into an area of dense tissue using a spring-loaded trigger mechanism.
19. The method of any of item 16-18, further involving filtering biological particles from exhaust air created by the vacuum.
20. The method of any of item 16-19, further involving recirculating the exhaust air into the vacuum to enhance vacuum levels.

As should be appreciated in view of the foregoing, a biopsy device, is disclosed. The biopsy device may include a manifold having a first passageway, a second passageway, and a third passageway, a fluid cartridge operably coupled to the manifold for providing fluid to the manifold, and a cutting mechanism operably coupled to the manifold. A handheld member may be slidably coupled to the manifold, and may be used to direct fluid between the first passageway, the second passageway, and the third passageway.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A biopsy device, comprising:
a manifold having a first passageway, a second passageway, and a third passageway;
a fluid cartridge operably coupled to the manifold for providing fluid to the manifold;
a cutting mechanism operably coupled to the manifold, the cutting mechanism comprising a cutting cannula, a sample notch, and a sample storage area; and
a handheld member slidably coupled to the manifold, wherein manual translation of the handheld member directs fluid between the first passageway, the second passageway, and the third passageway.

2. The biopsy device of claim 1, wherein the first passageway of the manifold comprises a first valve for creating a vacuum to introduce a tissue sample into the sample notch.

3. The biopsy device of claim 2, wherein the first valve comprises a rotational valve which may be manually rotated to control the vacuum created by the first valve.

4. The biopsy device of claim 2, wherein the first valve comprises a venturi valve.

5. The biopsy device of claim 2, wherein the, the second passageway of the manifold comprises a second valve for maintaining a vacuum in the sample notch and pushing the cutting cannula forward across the tissue sample.

6. The biopsy device of claim 5, wherein the second valve is a y-valve.

7. The biopsy device of claim 2, wherein the third passageway comprises a third valve for retracting the cutting mechanism and transporting the tissue sample to the sample storage area.

8. The biopsy device of claim 1, wherein the handheld member may be manually alternated between the first passageway, second passageway, and third passageway multiple times to obtain a plurality of tissue samples.

9. The biopsy device of claim 1, wherein the fluid cartridge is a carbon dioxide cartridge.

10. The biopsy device of claim 1, wherein the handheld member, the cutting mechanism, and the manifold are made of a transparent material.

11. The biopsy device of claim 1, wherein the manifold further comprises a fourth passageway, the fourth passageway pneumatically propels the cutting cannula into an area of dense tissue.

12. The biopsy device of claim 1, wherein the cutting mechanism further comprises a spring mechanism which propels the cutting cannula into an area of dense tissue.

13. The biopsy device of claim 1, further comprising a switch to temporarily disable the biopsy device as the handheld member slides between the third passageway and the first passageway.

14. The biopsy device of claim 1, further comprising a second fluid cartridge operably coupled to the manifold to provide fluid to the manifold.

15. A biopsy device, comprising:
a manifold having a first passageway, a second passageway, and a third passageway;
a fluid cartridge operably coupled to the manifold for providing fluid to the manifold;
a cutting mechanism operably coupled to the manifold, the cutting mechanism comprising a cutting cannula, a sample notch, and a sample storage area; and
a handheld member slidably coupled to the manifold, such that manual translation of the handheld member directs fluid between the first passageway, the second passageway, and the third passageway;
wherein the first passageway of the manifold comprises a first valve for creating a vacuum to introduce a tissue sample into the sample notch, the second passageway of the manifold comprises a second valve for maintaining the vacuum in the sample notch and pushing the cutting cannula forward across the tissue sample, and the third passageway comprises a pneumatic cylinder which retracts the cutting mechanism and transports the tissue sample to the sample storage area.

16. A method of operating a biopsy device, comprising:
providing fluid to a manifold of the biopsy device using a fluid cartridge;
manually sliding a handheld member of the biopsy device to a first position in the manifold;
creating a vacuum for capturing a tissue sample in the biopsy device;
manually sliding the handheld member of the biopsy device to a second position in the manifold;
pushing a cutting mechanism of the biopsy device across the tissue sample;
manually sliding the handheld member of the biopsy device to a third position;
retracting the cutting mechanism of the biopsy device such that the tissue sample is returned to a sample acquisition area of the biopsy device; and
returning the handheld member of the biopsy device to the first position.

17. The method of claim 16, further comprising creating the vacuum for capturing the tissue sample in the biopsy device using a venturi valve.

18. The method of claim 16, further comprising firing the cutting mechanism of the biopsy device into an area of dense tissue using a spring-loaded trigger mechanism.

19. The method of claim 16, further comprising filtering biological particles from exhaust air created by the vacuum.

20. The method of claim 19, further comprising recirculating the exhaust air into the vacuum to enhance vacuum levels.
